SEQUENCE LISTING (10 sequences)

SEQ ID NO: 1 - protein, 24 aa, Artificial Sequence
  Antimicrobial peptide alpha1alpha2
MAAFMKLIQFLATKGQKYVSLAWK

SEQ ID NO: 2 - protein, 20 aa, Artificial Sequence
  Antimicrobial peptide alpha2alpha3
QKYVSLAWKHKGTILKWINA

SEQ ID NO: 3 - protein, 26 aa, Artificial Sequence
  Antimicrobial peptide alpha3alpha4
KGTILKWINAGQSFEWIYKQIKKLWA

SEQ ID NO: 4 - protein, 34 aa, Artificial Sequence
  Antimicrobial peptide alpha1alpha2alpha3
MAAFMKLIQFLATKGQKYVSLAWKHKGTILKWIN

SEQ ID NO: 5 - protein, 35 aa, Artificial Sequence
  Antimicrobial peptide alpha2alpha3alpha4
KYVSLAWKHKGTILKWINAGQSFEWIYKQIKKLWA

SEQ ID NO: 6 - protein, 51 aa, Artificial Sequence
  Antimicrobial peptide R-NI01
MAAFMRLIQFLATRGQRYVSLAWRRRGTILRWINAGQSFEWIYRQIRRLWA

SEQ ID NO: 7 - protein, 28 aa, Artificial Sequence
  Antimicrobial peptide NT-205
MAAFMKLIQFLATKGQKYKSLAWKWKGL

SEQ ID NO: 8 - protein, 23 aa, Artificial Sequence
  Antimicrobial peptide H16
MAAFMKLIQFLATKGQKYINRKL

SEQ ID NO: 9 - protein, 18 aa, Artificial Sequence
  Antimicrobial peptide H1M
KLIQFLATKGQKLIQFLA

SEQ ID NO: 10 - protein, 51 aa, Artificial Sequence
  Antimicrobial peptide NI01
MAAFMKLIQFLATKGQKYVSLAWKHKGTILKWINAGQSFEWIYKQIKKLWA

(19) United States
(12) Patent Application Publication
(10) Pub. No.: US 2023/0100062 A1
(43) Pub. Date: Mar. 30, 2023

(54) ANTIMICROBIAL PEPTIDES

(71) Applicant: UNIVERSITY OF PLYMOUTH, Plymouth (GB)

(72) Inventors: Katharine HAMMOND, Teddington (GB); Mathew UPTON, Plymouth (GB); Maxim RYADNOV, Teddington (GB)

(21) Appl. No.: 17/765,976

(22) PCT Filed: Oct. 5, 2020
(86) PCT No.: PCT/EP2020/077878

§ 371 Date: Apr. 1, 2022

(30) Foreign Application Priority Data
Oct. 4, 2019 (GB) 1914347.8
Jul. 9, 2020 (GB) 2010572.2

(51) Int. Cl. C07K 14/195

(52) U.S. Cl. CPC C07K 14/195; A61K 38/00

(57) ABSTRACT

An isolated or recombinant polypeptide is provided and comprises a sequence selected from the group comprising NI01, α1α2, α2α3, α3α4, α1α2α3, α2 α3 α4, R-NI01, NT-205, H16, H1M NI01-R, 3NH (α1α3α3), H1 (α1α2), H2 (α2α3), 3HC (α2α3α4) or having at least 75% identity thereto, wherein the isolated or recombinant polypeptide is bactericidal and/or bacteriostatic.

### CROSS-REFERENCE TO RELATED APPLICATIONS

This application is a National Stage Entry of PCT/EP2020/077878 (filed Oct. 5, 2020), which cites the priority of United Kingdom Patent Application Numbers 2010572.2 (filed Jul. 9, 2020) and 1914347.8 (Oct. 4, 2019).

An electronic sequence listing (PCT/EP2020/077878.txt; size 4 kb; date of creation Nov. 7, 2022) submitted herewith is incorporated by reference herein in its entirely.

The present invention relates generally to antimicrobial peptides. Also described is a principle termed “flowering poration”—a multi-mode antibacterial mechanism encoded in a bacteriocin fold.

### INTRODUCTION

Host defense systems use pore-forming proteins to target pathogenic, host or aberrant cells.¹

Bacteria secrete such proteins to access nutrients from the cells of their hosts or outcompete other bacteria living in the same environmental niches,^{2,3} while human leukocytes release pore-forming proteins to kill pathogens.⁴ The spread of antimicrobial resistance has intensified interest in molecules promoting the lysis of microbial membranes with an emphasis on host defense peptides as potential anti-infectives.⁵ These peptides favour attack on microbial membranes and each tends to support one poration mechanism. The adoption of different mechanisms within the same sequence can be tuned by careful site-directed mutations.⁶ This modulation is possible because host defence peptides adopt relatively simple conformations in membranes. For example, only a single, short helix is required to elicit strong antimicrobial effects.² Bacteria themselves produce more complex antibacterial agents, termed bacteriocins, which specialize in killing closely related bacterial strains.⁷ The killing is proposed to occur through membrane poration, although experimental evidence for this conjecture has yet to be reported.⁸ Bacteriocins can be divided into subclasses according to their structural organisation and size;⁹ the most recent subclass is represented by a multi-helix bundle group. Bacteriocins of this subclass are small proteins comprising several α-helices packing into compact globular structures. Unlike other bacteriocins that have post-translational backbone, side-chain modifications or operate as tertiary complexes, proteins from this subclass are leaderless, single-chain and cysteine-free.^{3,10}

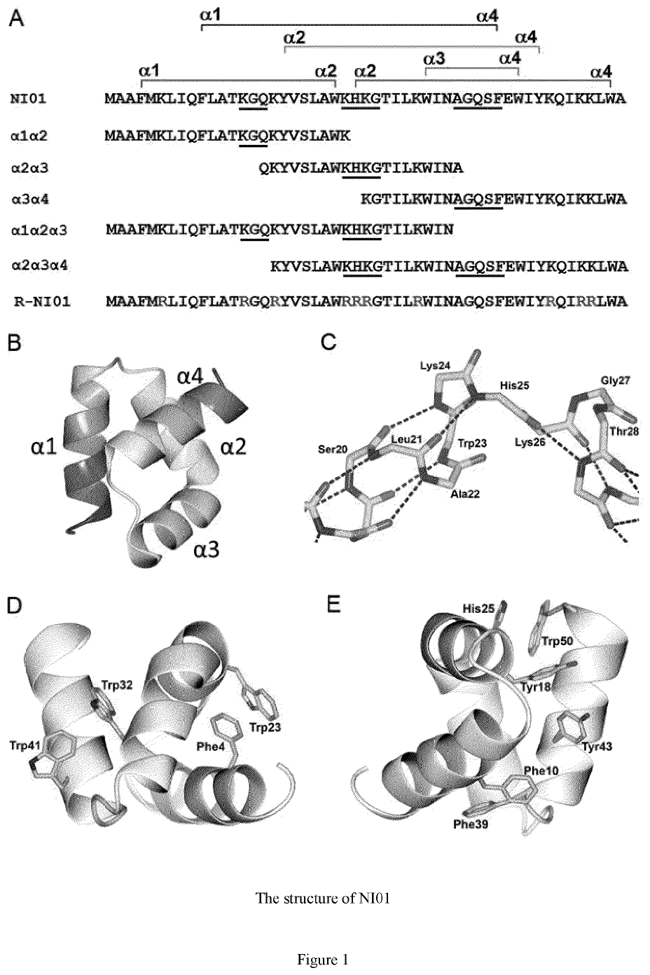

Given that their structures are multi-helix folds, we reason that such proteins must induce different modes of antimicrobial membrane disruption, with each mode supported by a specific constituent of the structure. Herein we validate this hypothesis, reporting the direct observation of multi-mode membrane disruption by bacteriocins. We first determine a high-resolution crystal structure of epidermicin NI01—a four-helix bacteriocin recently discovered in S. epidermis (FIG. 1A).¹¹ We then synthesise individual constituents of this structure—two- and three-helix hairpins (FIG. 1A and S1 in Supplemental Information)—characterise their biological and physical properties and compare them with those of the full-length epidermicin. Using atomic force microscopy, we demonstrate that each of helix-helix hairpins induces a distinct mode of membrane disruption in anionic phospholipid bilayers, whereas the intact protein combines all these modes into one synergetic mechanism which, to our knowledge, has not been observed before. We further demonstrate that this mechanism is not stereoselective as it is reproduced by the all-D version of epidermicin. We show that all tested structures are appreciably antimicrobial and that synergy between the different corresponding modes of membrane disruption balances out the antibacterial and hemolytic activities of the protein. Finally, we compare the disruption mechanisms of epidermicin and another bacteriocin from the same fold group and find that the two mechanisms are strikingly similar sharing the same disruption modes.

SUMMARY: bacteriocins are a distinct family of antimicrobial proteins postulated to porate bacterial membranes. However, direct experimental evidence of pore formation by these proteins is lacking. Here we report a multi-mode poration mechanism induced by four-helix bacteriocins, epidermicin NI01 and aureocin A53. Using a combination of crystallography, spectroscopy, bioassays and nanoscale imaging, we established that individual two-helix segments of epidermicin retain antibacterial activity but each of these segments adopts a particular poration mode. In the intact protein these segments act synergistically to balance out antibacterial and hemolytic activities. The study sets a precedent of multi-mode membrane disruption advancing the current understanding of structure-activity relationships in pore-forming proteins.

KEYWORDS: bacteriocins, antimicrobial resistance, nanoscale imaging, protein crystallography

Some aspects and embodiments may be based on a principle of flowering poration—a synergistic multi-mode antibacterial mechanism by a bacteriocin fold.

An aspect of the present invention provides an isolated or recombinant polypeptide comprising a sequence as described herein, or having at least 75% identity thereto, wherein the isolated or recombinant polypeptide is bactericidal and/or bacteriostatic.

The isolated or recombinant polypeptide may comprise a sequence selected from the group comprising or consisting of:

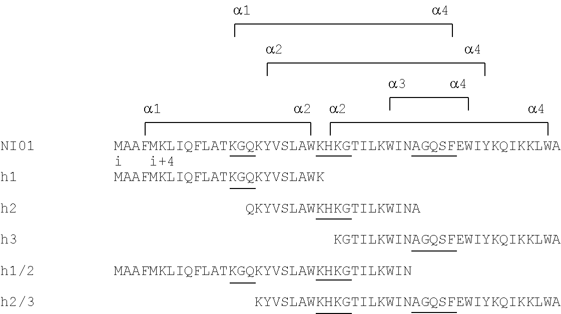

NI01

α1α2

α2α3

α3α4

α1α2α3

α2α3α4

R-NI01

NT-2-5

H16

H1M

And/or from the group comprising or consisting of:

NI01

A53

| | |
|---|---|
| NI01 | 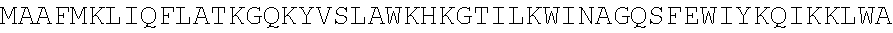 |
| A53 | 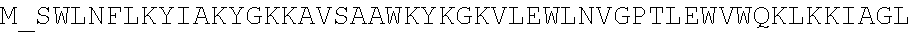 |

And/or from the group comprising or consisting of:

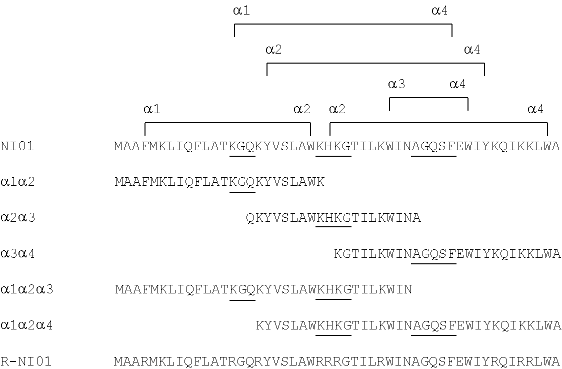

| | |
|---|---|
| NI01 | MAAFMKLIQFLATKGQKYVSLAWKHKGTILKWINAGQSFEWIYKQIKKLWA 51 |
| NI01-R | MAAFMRLIQFLATRGQRYVSLAWRRRGTILRWINAGQSFEWIYRQIRRLWA 51 |
| NT-205 | MAAFMKLIQFLATKGQKYKSLAWKWKGL 28 |
| H16 | MAAFMKLIQFLATKGQKYINRKL 23 |
| HIM | KLIQFLATKGQKLIQFLA 18 |
| 3NH (α1α3α3) | MAAFMKLIQFLATKGQKYVSLAWKHKGTILKWIN 34 |
| Hl (α1α2) | MAAFMKLIQFLATKGQKYVSLAWK 24 |
| H2 (α2α3) | QKYVSLAWKHKGTILKWINA 20 |
| 3HC (α2α3α4) | KYVSLAWKHKGTILKWINAGQSFEWIYKQIKKLWA 35 |

The polypeptide may have at least 90% identity to a sequence described herein.

The present invention provides for a composition consisting of one sequence type, and also provides for a combination of two or more the sequences.

The present invention also provides an isolated or recombinant bacteriocin-based polypeptide sequence that defines a plurality of helical hairpins when in a folded configuration, in which each hairpin provides a distinct mode of membrane disruption.

The sequence may be based on a four-helix bundle bacteriocin.

The present invention also provides an isolated or recombinant polypeptide sequence that provides a plurality of distinct bacterial membrane disruption modes which combine in use to provide one synergistic mechanism of poration.

The present invention also provides a fold-regulated, multi-mode poration polypeptide sequence, said sequence being bactericidal and/or bacteriostatic.

The present invention also provides a bactericidal and/or bacteriostatic polypeptide or polypeptide combination comprising sequence/s that define two or more helical hairpin types, in which each helical hairpin type provides a different mode of membrane disruption.

The hairpin types may be provided within the same sequence.

The hairpins may be two helix or three helix hairpins or four helix hairpins.

The present invention also provides an isolated or recombinant nucleic acid sequence comprising a sequence encoding the polypeptide/s as described herein.

The present invention also provides a pharmaceutical composition comprising one or more of the polypeptides as described herein.

The pharmaceutical composition may be for the treatment of a bacterial infection.

The present invention also provides an anti-microbial formulation comprising one or more of the polypeptides as described herein.

The present invention also provides an isolated or recombinant polypeptide comprising a sequence described herein, or having at least 75% identity thereto, wherein the isolated or recombinant polypeptide is bactericidal and/or bacteriostatic.

Different aspects and embodiments can be used together or separately.

Embodiments of the present invention are more particularly described, by way of non-limiting example, herein.

The example embodiments are described in sufficient detail to enable those of ordinary skill in the art to embody and implement the systems and processes herein described. It is important to understand that embodiments can be provided in many alternate forms and should not be construed as limited to the examples set forth herein.

Embodiments can be modified in various ways and take on various alternative forms. There is no intent to limit to the particular forms disclosed. On the contrary, all modifications, equivalents, and alternatives falling within the scope of the appended claims should be included.

Unless otherwise defined, all terms (including technical and scientific terms) used herein are to be interpreted as is customary in the art. It will be further understood that terms in common usage should also be interpreted as is customary in the relevant art and not in an idealised or overly formal sense unless expressly so defined herein.

One of ordinary skill in the art will appreciate the many possible applications and variations of the present invention based on the following examples of possible embodiments of the present invention.

### RESULTS

Epidermicin Folds into a Four-Helix Bundle Topology

FIG. 1. The structure of NIO1. (A) Primary structure of NI01 and its derivatives—two-helix and three-helix hairpins, and an arginine mutant, R-NI01. Coloured staples indicate π-π interactions between aromatic residues of different helices, labelled α1-α4. Turns are underlined in the sequences. Arginine residues in RNI01 are shown in blue. (B) Crystal structure of NI01. Ribbon representation from the N-terminus (blue) to the C-terminus (red). (C) Stick representation of the central kink linking two terminal hairpins at H25. (D) Two aromatic pairs, F4-W23 and W32-W41, between sequential helices: α1α2, and α3α4, respectively. (E) Remaining three aromatic pairs, all involving the C-terminal helix, H25-W50, Y18-Y43 and F10-F39.

The X-ray structure of NI01 revealed that it folds into a compact, four-helix bundle in which two α-hairpins are linked through a kink (φ=−116° and ψ=36°) in the central helix at H25 (FIG. 1B, C). The transition between α1 and α2 is mediated by a type III β-turn, and from α3 to α4 by G36, which forms a break at the end of the third helix (FIG. 1B, Table S1). The hydrophobic residues of all helices are buried in the core of the bundle, which is characteristic of bacteriocins and essential to stabilise the fold in solution. Aromatic residues account for 20% of all residues in this protein but are not engaged in the core. Instead, their side chains are locked in paired π-π interactions that appear to act as staples between spatially adjacent helices. Five pairs are formed to support interhelical crossovers, only two of which are formed between sequential helices, namely the F4-W23 and W32-W41 pairs that link α1 and α2, and α3 and α4 helices, respectively (FIG. 1D). Four of the pairs involve the C-terminal helix (α4) including all of the remaining pairs, H25-W50, Y18-Y43 and F10-F39 (FIG. 1E). Given that this helix is stapled with each of the other three helices, it may function as a leader helix, which synchronizes the insertion of NI01 into membranes. The central α2 and α3 helices share no aromatic pairs between them, which is expected for helices oriented perpendicular to one another, and is common for leaderless bacteriocins.¹² Finally, the analysis of the structure by PISA13 did not indicate any significant contacts between protein monomers indicating that the protein is monomeric in aqueous solution (FIG. 1B).

### Epidermicin Folds Cooperatively in Solution and Binds Strongly to Anionic Membranes

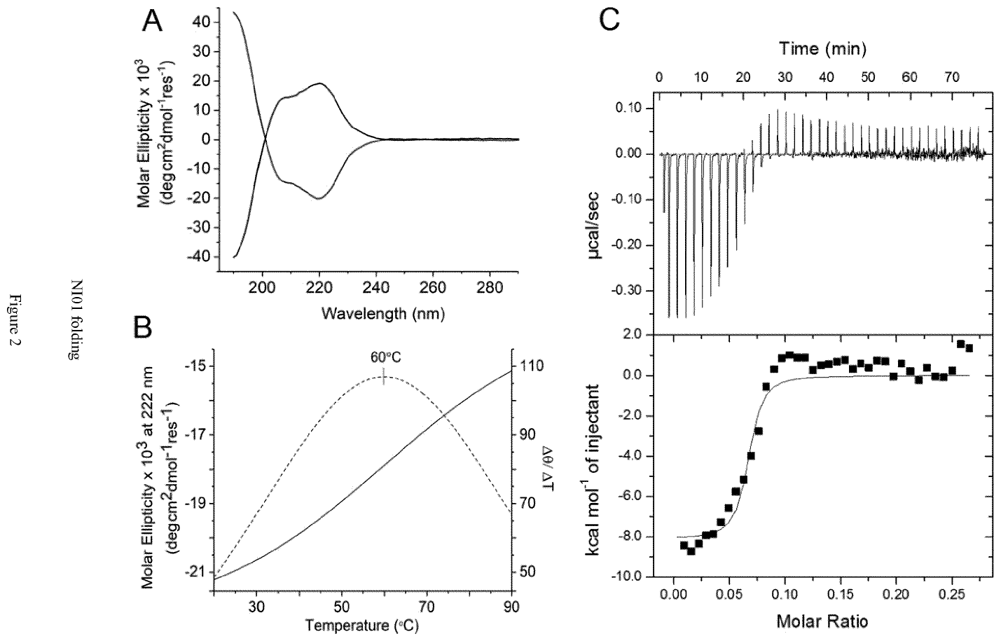

Each helix in NI01 is at least two helical turns in length, which is sufficient to support the cooperative folding of the protein. Circular dichroism (CD) spectroscopy confirmed helix formation by NI01 in aqueous buffers (FIG. 2A), with sigmoidal unfolding curves giving a single transition midpoint (TM) of ˜60° C. (FIG. 2B).

FIG. 2. NI01 folding. (A) CD spectra for NI01 (blue line) and its all-D form (20 μM protein) in 10 mM phosphate buffer (black line). (B) Thermal unfolding curve and its first derivative highlighting a single transition point (TM). (C) Isothermal titration calorimetry of NI01 (500 μM) binding to bacterial mimetic membranes. Heat absorbed (μcal/s) for each isotherm is plotted versus titration time (upper panel). Integrated heats (kcal/mol) are plotted versus protein-lipid molar ratios (lower panel), showing a curve fitting to a one-set binding model (black line).

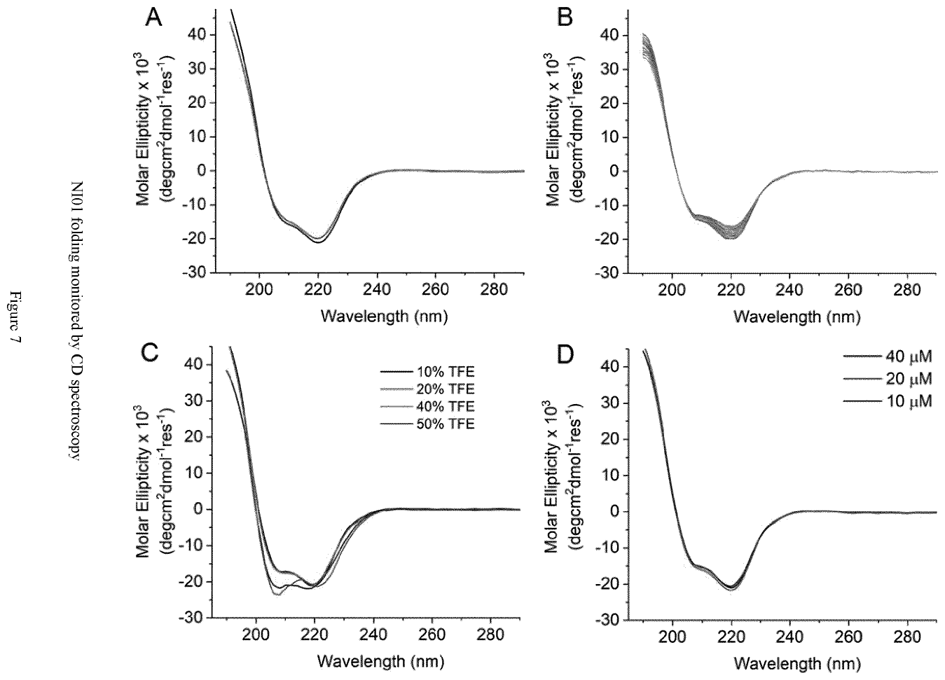

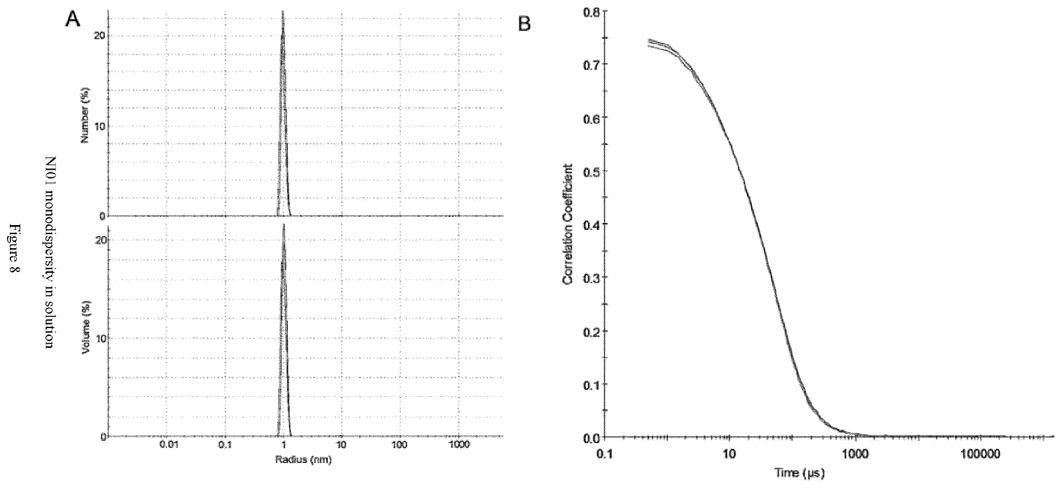

Denaturation was also fully reversible: the spectra collected before and after the thermal denaturation were nearly identical (FIG. 7A). The signal intensity at 202 nm, which remained the same during denaturation provided a clear isodichroic point indicating a two-state transition between helical and unfolded forms (Fig S2B). However, even at temperatures as high as 90° C. NI01 retained helical content: the spectral Δε222/Δε208 ratios for all spectra recorded during the thermal transition were ≥1, as expected for helical bundles (FIGS. 2A and 7B).¹⁴ The observation is consistent with the fact that NI01 retains antimicrobial activity following exposure to elevated temperatures (80° C.), as reported elsewhere.⁹ The helical content of the protein in aqueous buffers was comparable to that in aqueous 2,2,2-trifluoroethanol (TFE) (FIG. 7C). Fluorinated alcohols promote intramolecular hydrogen bonding by excluding water from the solute and encompassing the polypeptide chain in a hydrophobic “matrix”.15 Thus, the TFE-induced helix formation shows the extent to which an individual chain can fold into a helical state excluding supramolecular contributions. With no apparent changes at different TFE concentrations (FIG. 7C), the helical content of NI01 was also independent of peptide concentrations (FIG. 7D). Collectively, the results are indicative of a highly stable protein that is fully folded in solution. Similar to other pore-forming proteins, which target bacteria, epidermicin is cationic having a net charge of +8 at neutral pH. In the crystal structure of NI01, polar side chains of each helix cluster on the exterior of the protein. In solution, the protein is a monodisperse particle of 2 nm in diameter exhibiting a high surface charge (ζ-potential of 20.8±3.8 mV). These characteristics confer a high stability on the protein, allowing it to bind to anionic bacterial membranes as a monomer (FIG. 8).

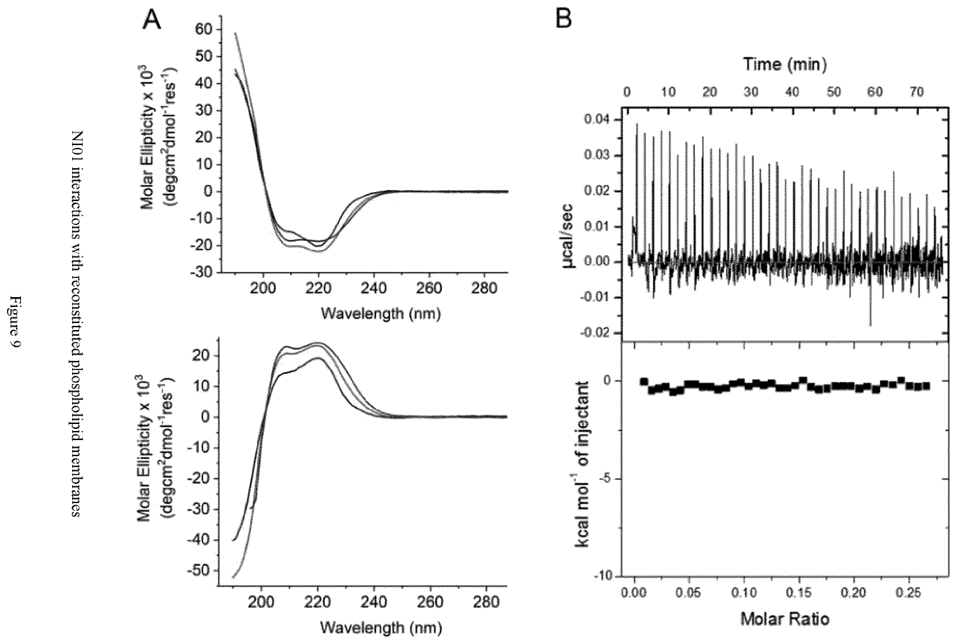

Since NI01 is already folded in solution, CD spectroscopy could only reveal additive changes in helicity in membranes. As expected, the helical content for NI01 remained unchanged when it was measured in reconstituted phospholipid bilayers, which were constructed as unilamellar vesicles to mimic bacterial (anionic) and mammalian (zwitterionic) membranes (FIG. 9A). Isothermal titration calorimetry (ITC) provided a more quantitative measure of protein-membrane interactions. Measured by titrating NI01 into anionic phospholipid membranes, binding isotherms revealed an exothermic process indicating enthalpy-driven ionic and hydrogen-bond interactions (FIG. 2C). As protein-lipid ratios increased endothermic processes became more pronounced suggesting increasing contributions from hydrophobic interactions. This can be attributed to that the protein inserts deep into the hydrophobic interface of the bilayer (FIG. 2C). The integrated heats fitted into a single site binding model gave a dissociation constant (KD) of 0.3 μM with a ΔG of −8.9 kcal/mol, both values consistent with the characteristics of membrane-targeting antibiotics and pore-forming proteins.^{16, 17} The biphasic binding found during the titrations suggests a synergistic, multi-mode mechanism by which NI01 selectively targets bacterial membranes. No binding was detected in zwitterionic phospholipid membranes (FIG. 9B), consistent with negligible levels of toxicity towards mammalian cells lines¹¹ and erythrocytes (Table S2). It can thus be concluded that the protein selectively disrupts bacterial membranes by binding to their surfaces through charge interactions and then re-arrangement into pores or channels.

### Epidermicin Induces a Synergistic, Multi-Mode Poration Mechanism in Anionic Membranes

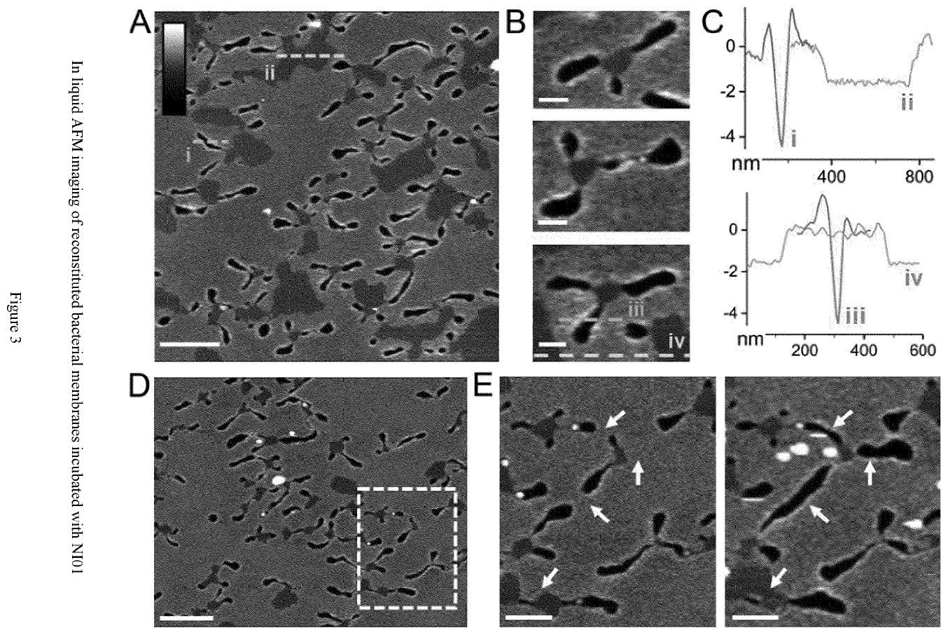

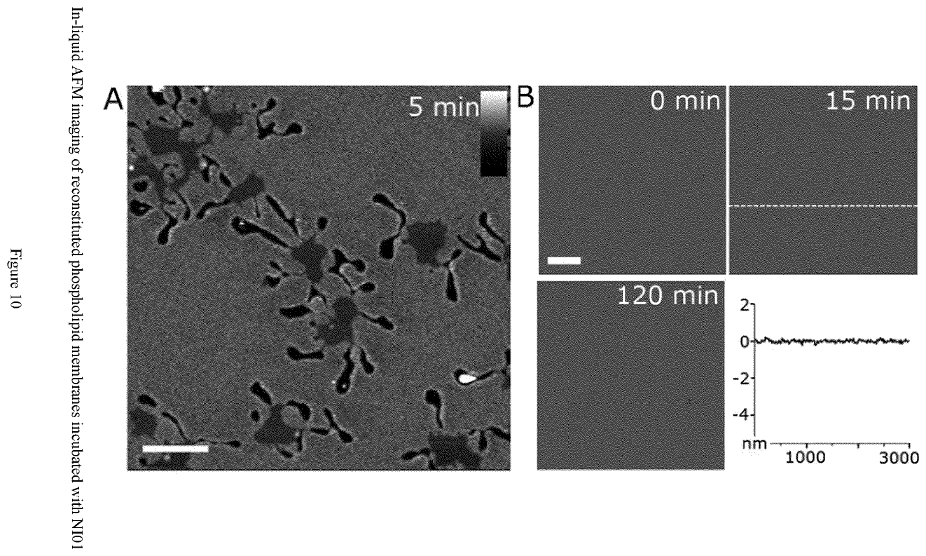

We probed the mechanism of membrane disruption by visualizing the effect of NI01 on reconstituted membranes using time-resolved atomic force microscopy in aqueous buffers (in liquid AFM). The membranes of the same lipid composition used for the biophysical measurements in solution were deposited on mica surfaces as supported lipid bilayers (SLBs).¹⁸ The resulting preparations yield flat (to within ≤0.1 nm) fluid-phase membranes that allow for accurate depth measurements of surface changes.^{19,20} Within minutes NI01 formed floral patterns on the SLBs. These patterns comprised roughly circular patches of thinned membranes radially propagating with petal-like lesions or pores (FIG. 3A, 10A). Most patterns had three petals per patch (FIG. 3B). The patches were ˜2 nm in depth half-way through the bilayer, which is consistent with membrane thinning effects commonly observed for antimicrobial peptides.⁶ In contrast, the petal-like lesions extended all the way across the membrane (4 nm), i.e. were transmembrane pores (FIG. 3C). The lesions were tapered at one end connecting with their respective patches, whereas the opposite end appeared as a growing circular pore merging with other pores (FIGS. 3D, E and 10A). Complementary to the ITC results, the AFM measurements showed that the bacteriocin was selective towards bacterial membranes. No changes could be detected in SLBs mimicking mammalian membranes, even at higher concentrations (FIG. 10B).

FIG. 3. In liquid AFM imaging of reconstituted bacterial membranes incubated with NI01. (A) Topography of NI01-treated SLBs mimicking bacterial membranes (see Methods). (B) Higher magnification images of individual patches (brighter areas) with petal-like pores (darker areas) from (A). The images were taken within the first 10 min of incubation with NI01 (0.25 μM). (C) Height profiles as measured along the highlighted lines in (A) and (B). (D) SLBs imaged at a low magnification, with the framed area imaged at a higher magnification (E) over 1 hour to show growing pores and patches as highlighted by white arrows (from left to right). Colour scale bar is 15 nm. Length scale bars are 500 nm for (A) and (D), 100 nm for (B) and 200 nm for (E).

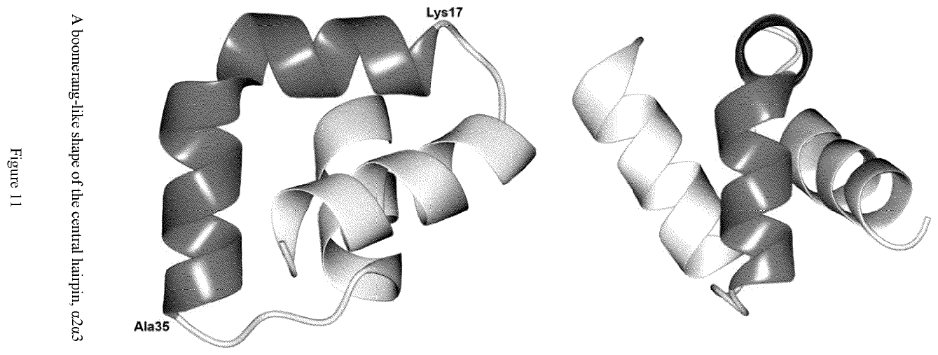

The patches of thinned membranes appear as contact regions from which NI01 radially diffuses into the lipid matrix. This scenario resembles mechanisms proposed for four- and five-helix protein toxins that insert into the upper leaflet of the bilayer where they arrange into pores.^{21,22} Similarly, antimicrobial peptides accumulate in the upper leaflet causing the thinning of phospholipid bilayers.²³ These studies indicate that as more peptide binds to the bilayers thinning areas grow in size but not in depth, as also observed for NI01 (FIG. 3E).²⁴ This suggests that a portion of NI01 should specialize in binding to the upper leaflet and be plastic enough to orchestrate protein reassembly into pores. β-hairpins and bent α-helices are common folding topologies that induce membrane thinning and exfoliation.^{25, 26} NI01 has three overlapping helical hairpins (FIG. 1A). The two terminal hairpins have similar up-and-down topologies, in which individual helices are clearly separated by extended turns (FIG. 1B). With the N- and C-terminal helices being twice the length of the central helices, the terminal hairpins have the capacity for transmembrane insertion. In contrast, the two central helices are arranged into an α-α corner via a kink at an obtuse angle, which constrains the helices into a more open hairpin conformation (FIGS. 1B and 11). A boomerang-like shape of this hairpin could make it lie flat on membrane surfaces, favouring membrane thinning over transmembrane poration (FIG. 11).

### Each Mode of the Membrane Disruption Mechanism is Activated by a Specific Two-Helix Constituent of Epidermicin

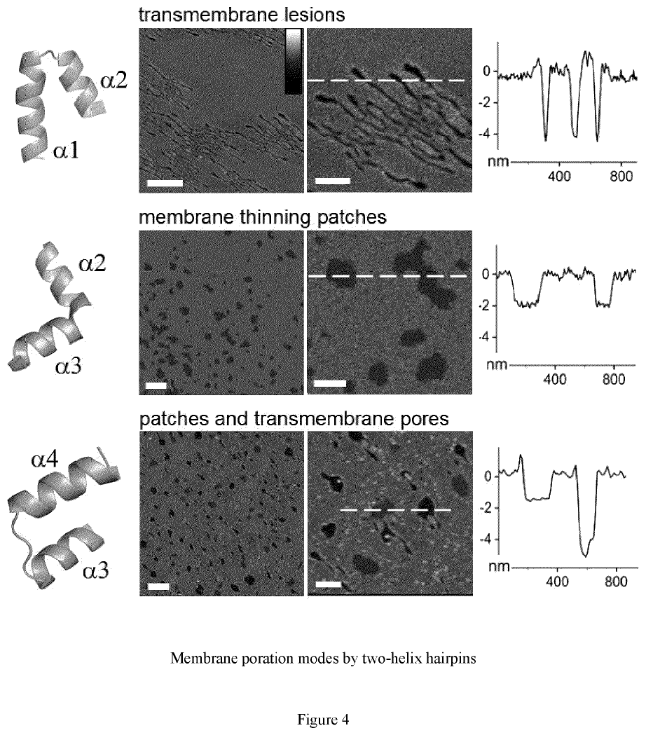

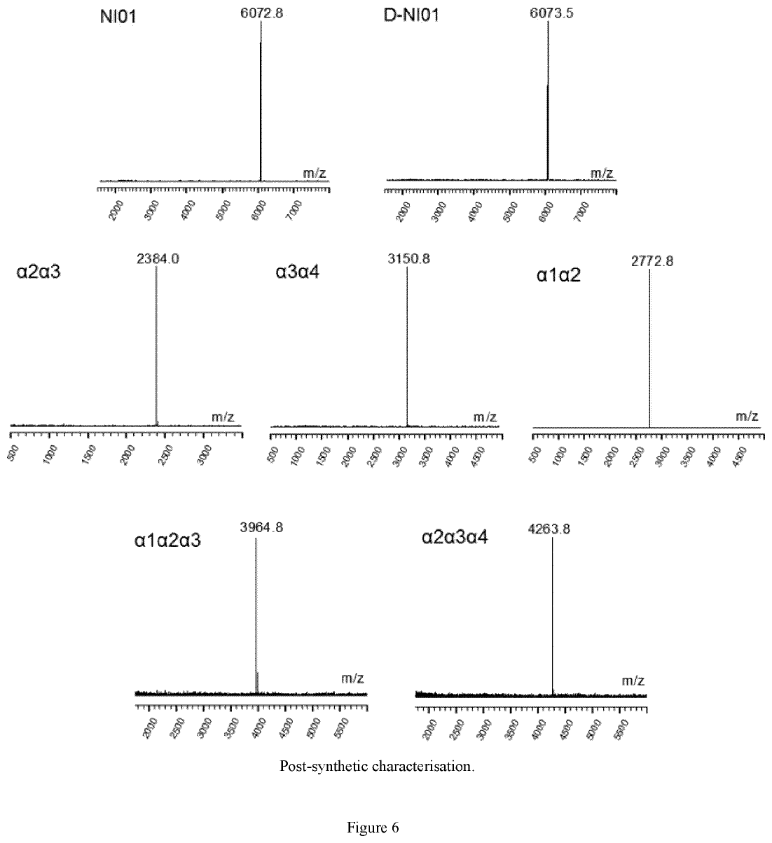

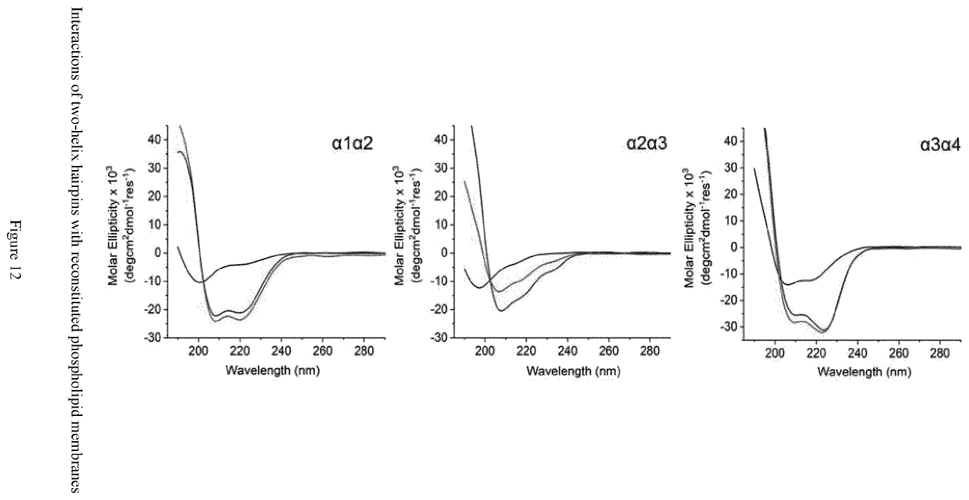

To gain more insight into these predictions, all three hairpins—α1α2, α2α3 and α3α4 (FIG. 1A), were synthesised (FIG. 6), characterised (FIG. 12) and imaged by AFM on SLBs (FIG. 4). The first two hairpins showed strikingly distinctive behaviours, each supporting exclusively one mode of the mechanism observed for NI01 (FIG. 4). The first hairpin, α1α2, formed extended petal-like pores that ran parallel to each other without branching. The regions of thinned membranes that in NI01 served as branching points for the pores were absent in SLBs treated with α1α2. In contrast, membrane thinning was apparent in SLBs treated with α2α3, with no indication of transmembrane pores. Although the regions imaged for α2α3 were similar in size and morphology to those formed by NI01, the petal-like pores of α1α2 appeared thinner and more extended when compared to those of NI01 (FIG. 4). Wide, circular pores were dominant in SLBs treated with α3α4, with membrane-thinning patches being also abundant, which together indicate that α3α4 induced a mixed mode of membrane disruption (FIG. 4).

FIG. 4. Membrane poration modes by two-helix hairpins. In liquid AFM topography images of SLBs mimicking bacterial membranes treated with two-helix hairpins derived from NI01. The images were taken within the first 5 min of incubation with each hairpin (0.25 μM). Height profiles are measured along the highlighted lines. Colour scale bar is 15 nm, length scale bars are 500 nm for the low magnification images (left) and 200 nm for the high magnification images (right).

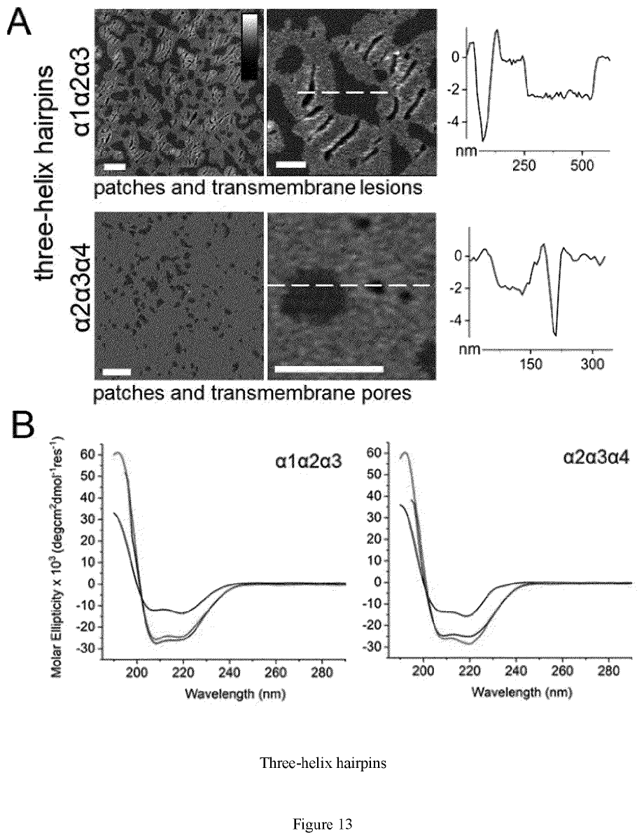

In these experiments, it is evident that membrane thinning patches occur only when α3 is present (FIG. 4). Both α2α3 and α3α4 incorporate this helix and α3α4 is the only of the three hairpins that induces the two membrane rupture modes. Thus, α3 appears to support the interplay of rupture modes favoured by other helices. Further evidence for this was derived from the behaviour of the two terminal three-helix hairpins, which were also produced as individual sequences (FIG. 6). The N-terminal hairpin (α1α2α3) should combine two rupture modes: transmembrane lesions of α1α2 and thinned patches of α2α3, but without the synergy characteristic of NI01 manifesting in the conserved combined patterns of thinned patches and petals. For the C-terminal three-helix hairpin (α2α3α4) membrane thinning is expected to dominate as the synergy was already lacking in α3α4, and α2α3 did not form transmembrane pores. Consistent with this reasoning, the two predicted modes of membrane disruption were evident for α1α2α3 (FIG. 13A). Although circular transmembrane pores could be detected for α2α3α4, these were much smaller in size, which contrasted with the abundance of thinned membrane regions caused by this hairpin (FIG. 13A). The two three-helix hairpins were partially folded in solution, indicating impaired cooperativity of folding in solution when compared to that of NI01 (FIG. 13B). Comparable helical content in solution was recorded for α3α4, which is notable given that α1α2 and α2α3 were unfolded (FIG. 12). As for these two-helix hairpins, helicity sharply increased upon membrane binding for the terminal hairpins (FIGS. 12, 8B) The results indicate that two- and three-helix hairpins containing α3 form membrane thinning patches, which emphasizes the mediatory role of this helix in supporting the interplay of the different modes of membrane disruption.

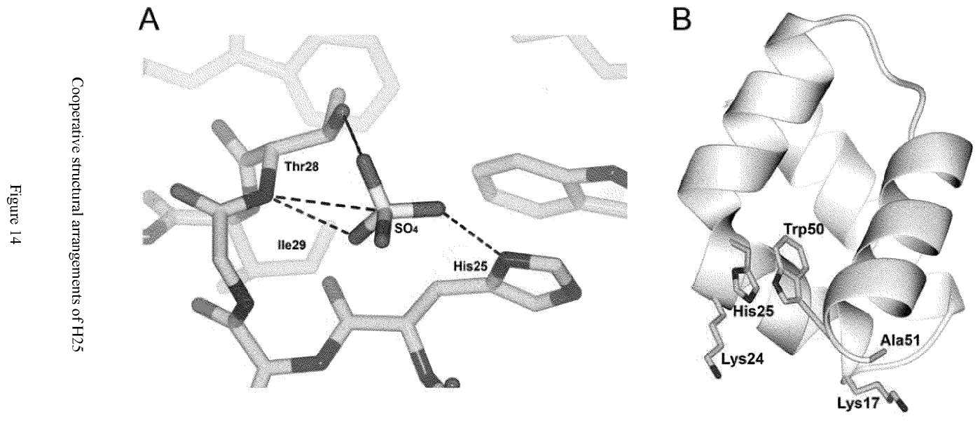

The C-terminal helix, α4, is the only helix in NI01 interacting with all other helices via the aromatic pairs. It is also a part of α3α4, which is the only two-helix hairpin that folds in solution (FIG. 1). In α2α3α4, α2 and α3 share no single aromatic pair between them. H25 is an exception in that it is located in the central turn connecting the two helices. The residue forms an aromatic pair with the terminal W50, which appears important for directing the insertion of α4. In addition, H25 is cationic, suggesting that it may bind to anionic lipids. Indeed, in both crystal forms H25 was observed to bind to SO2-4 (FIG. 14). In antimicrobial peptides similar electrostatic interactions are formed between phosphate groups and cationic residues, which in NI01 are represented by lysine (FIG. 1A). Consistent with the exothermic phase in the ITC measurements (FIG. 2C), the residue displaces water from the phosphate and strongly binds to it. The formed interactions are strong enough for membrane binding and cooperative enough to allow different disruption modes to manifest in synergy, one distinctive, conserved mechanism.

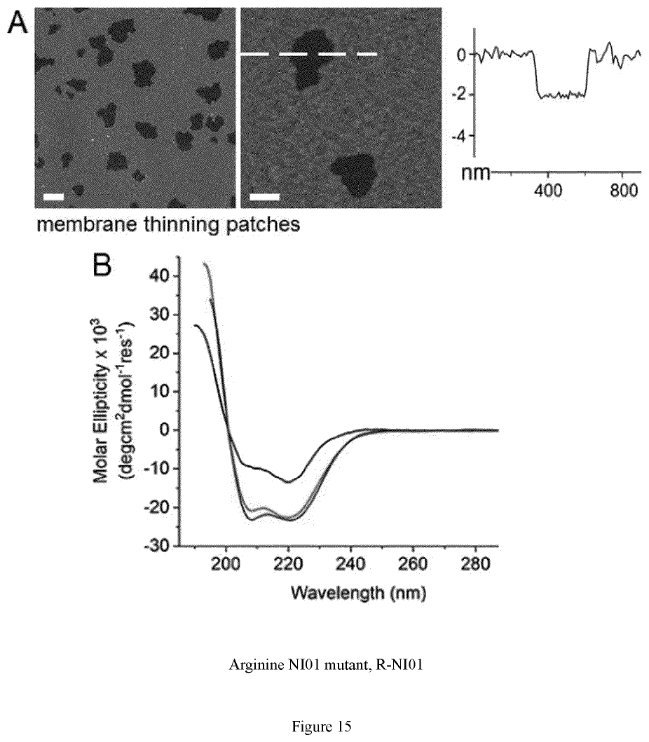

To test these conventions, all lysines were replaced with arginines in an all-arginine mutant of NI01, R-NI01 (FIG. 1A). Unlike lysine, arginine is positively charged at all stages of membrane binding and insertion, and traps more phosphate and water by providing five hydrogen-bond donors.²⁷ This difference manifests in a tighter binding to membrane surfaces, and, as shown elsewhere, limits protein insertion into the upper leaflet of the bilayer.²⁶ Replacing H25 with arginine preserves the positive charge in the site, but also eliminates the H25-W50 pair compromising cooperativity in interactions between helices and the ability of α4 to insert. Indeed, this mutant produced exclusively thinning patches in the membranes, which were strikingly similar to those observed for α2α3 (FIGS. 4 and 15A). Furthermore, R-NI01 was 50% less helical than NI01 (FIG. 15B). The loss in helicity was restored upon binding to phospholipid membranes (FIG. 15B). This behaviour was similar to that of the three-helix hairpins, which were considerably less helical in solution than NI01, but whose helical content increased in membranes (FIG. 13B). These results indicate that this mutation had a detrimental effect on NI01 folding in solution and its multimode mechanism in membranes. The importance of these findings is two-fold. Firstly, the analysis of disruption mechanisms by individual hairpins confirm that NI01 exhibits a conserved, synergistic mechanism of membrane disruption. This is ensured by the cooperative folding of NI01 and tertiary contacts of its constituent helices. Each of these helices makes an important contribution to the complex pattern of this mechanism, but none of them is sufficient individually. Secondly, all hairpin derivatives disrupt bacterial mimetic membranes. This suggests that all of the hairpins are antimicrobial and that their antimicrobial activities do not require a specific receptor to target bacteria, and therefore the antimicrobial activity of NI01 is not stereoselective.

### Synergy in the Multi-Mode Mechanism Determines the Biological Selectivity of the Protein

Considering the first point, NI01 and all of its derivatives exhibited comparable levels of antibacterial activity. Minimum inhibitory concentrations (MICs) were similar to those obtained for conventional antibiotics (Table S2). Noteworthy differences were observed in MICs for Gram positive S. aureus and Gram-negative P. aeruginosa. NI01, α1α2 and α3α4 were equally effective against S. aureus and ineffective against P. aeruginosa. Intriguingly, α2α3 showed a reversed trend, which may be attributed to differences in the cell-wall structure of the bacteria. The peptidoglycan layer of Gram-positive cells is rich in anionic teichoic polymers, which might prevent α2α3 from reaching the cytoplasmic membrane.28 This proposition is supported by the observation that α2α3 remained largely unfolded in membranes and hence is subject to conformational fluctuations caused by binding to the teichoic polymers (FIG. 12). All other hairpins and R-NI01 responded to membrane binding with sharp increases in helicity. Other Gram-positive bacteria, B. subtilis and M. luteus, proved to be susceptible to all of the NI01 derivatives used (Table S2). Peptidoglycans in these bacteria undergo continuous transformations from thick to thin layers, which makes their membranes more vulnerable to the attack by α2α3.29,30 Consistent with the lack of activity against S. aureus, α2α3 failed to affect methicillin-resistant S. aureus (MRSA) strains. NI01 and the other two-helix hairpins maintained similar levels of activity against these pathogens when compared to those for the susceptible strain (Tables S2&3). The three-helix hairpins were less active against MRSA. Both these hairpins incorporate α2α3 that was inactive against any of the S. aureus strains tested. Therefore, the impact of thicker peptidoglycan layers of MRSA,31 on their activity is expected to be greater (Tables S2& S3). Another notable trend was observed for Gram-negative bacteria. NI01 and its derivatives appeared to be active only against E. coli. Similar to peptidoglycan layers in Gram-positive bacteria, lipopolysaccharide (LPS) layers represent a key virulence factor for Gram negative membranes. To probe this, two additional E. coli strains were tested: a short-chain LPS or rough strain, SBS363, and a smooth strain comprising full-length, mature O-chains, ML35.32 All derivatives were active against the rough, more susceptible type, but the smooth type was resistant to all two-helix hairpins, except α1α2 (Table S3).

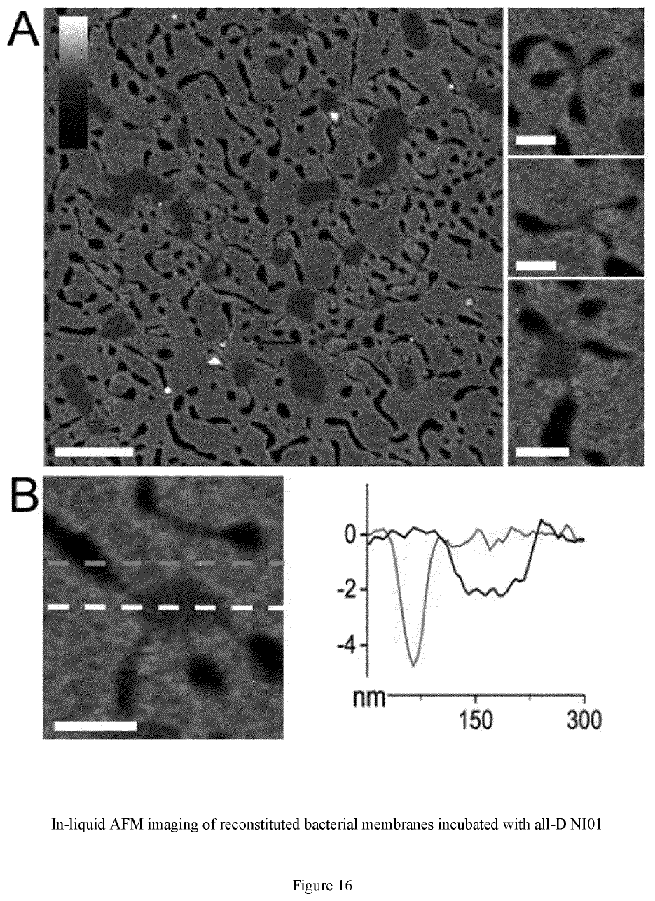

Considering the second point, NI01 was re-made into an all-D form (FIG. 6). The protein adopted helical conformations that quantitatively mirrored those of the wild-type all-L NI01 in both solution and membranes (FIGS. 2A and 9A). In bacterial membranes the all-D form revealed a strikingly similar pattern to that of the all-L form (FIG. 16), and both epimeric forms exhibited comparable antibacterial activities across all bacteria and strains tested (Tables S2 & S3). Taken together the results of these biological tests confirmed the antibacterial properties of NI01, with stronger activities observed for the derivatives exhibiting transmembrane disruption modes.

Bacteriocins, unlike host defence peptides or helminth defence molecules,33 do not originate from multicellular organisms. However, there can be a selective pressure on bacteria residing in human hosts to remain in a commensal state. Consequently, bacteriocins produced by these bacteria should be able to differentiate between bacterial and host cells. For therapeutic applications, this requirement extends to red blood cells, which are weakly anionic and can also be targeted by bacteriocins. In this regard, NI01 proved to be non-hemolytic in both L- and D-forms at concentrations equivalent to >100×MICs against Gram positive strains. This result was striking as all other derivatives caused appreciable hemolysis, except α2α3, which showed no hemolytic activity even at high concentrations (>600 μg/mL). These findings suggest that this hairpin rebalances antibacterial and hemolytic activities of NI01 by effectively diminishing the impact of the terminal helices, which favour transmembrane poration. Hemolytic activities drastically increased for R-NI01 and other hairpins, all of which lack the synergy of inter-helix interactions characteristic for NI01. As a consequence, these derivatives were incapable to differentiate between bacterial and erythrocytic membranes.

Mechanistic Similarities with Other Four-Helix Bacteriocins

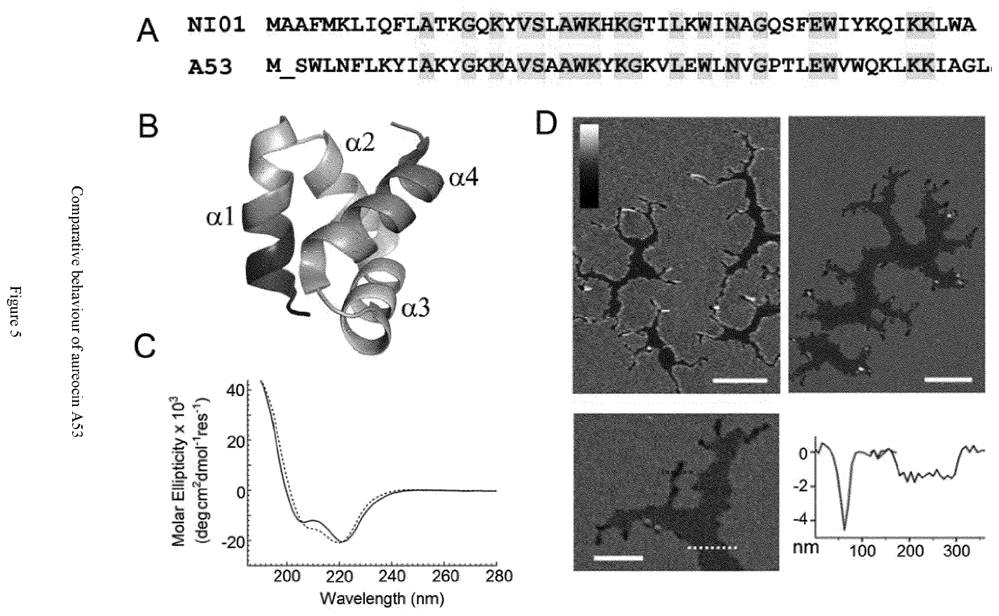

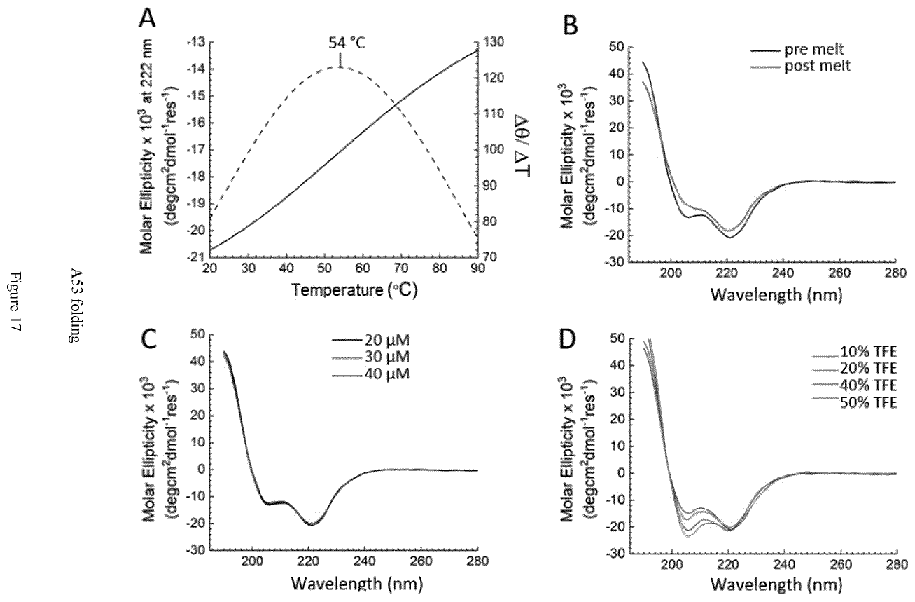

To this end, we have shown that NI01 exhibits a unique multi-mode mechanism of membrane disruption. To the best of our knowledge, this is also the first direct observation of bacteriocin induced poration, which prompts an obvious comparison with other bacteriocins. With this in mind, we performed a similar analysis for aureocin A53 (FIG. 5A). This bacteriocin belongs to the same four-helix bundle group and its structure was recently solved by NMR spectroscopy (FIG. 5B).³⁴ As gauged by CD spectroscopy, the protein folded remarkably similar to that of NIO1, with the two proteins having a nearly identical helical content (FIG. 5C). A53 was as stable as NI01 with (TM) of ˜54° C. (FIG. 17A), folded reversibly and independently of concentration (FIG. 17B, C), and showed no changes at increasing TFE concentrations (FIG. 17D). BLAST searches indicated a significant level of sequence homology between the two proteins (38% identity). The location and extent of turn regions and individual helices were also very similar, while hydrophobic, polar and aromatic residues were well conserved (FIG. 5A). Outside of the identity regions the exact sequence compositions of NI01 and A53 are different. Despite that the observed structural similarities suggest that A53 might exhibit a similar mechanism of membrane disruption.

FIG. 5. Comparative behaviour of aureocin A53. (A) Amino-acid sequences of NI01 and A53. Identical amino acids are highlighted in cyan. (B) NMR solution structure of A53 bacteriocin (PDB entry 2N8O rendered by PyMol).34 (C) CD spectra for NI01 (dashed line) and A53 (black line) (20 μM protein) in 10 mM phosphate buffer. (D) Topography AFM images of anionic SLBs treated with A53 (0.25 μM), and height profiles measured along the highlighted lines. Colour and length scale bars are 15 nm and 500 nm, respectively.

AFM analyses of A53-treated anionic membranes showed disruption modes similar to those recorded for NIO1: membrane thinning patches and transmembrane lesions and pores (FIG. 5D). The patches were more extended than those for NI01. The petal-like lesions were morphologically similar to those of NI01, also ending with circular pores and grew out of the patches. Depth profiles for each mode were identical for the two bacteriocins. Overall, the same characteristics of membrane disruption were evident for both proteins, which exhibited the same folding topology, sequence length and helical content. The variations in the mechanisms may be attributed to amino acid permutations in helical and turn regions of the two proteins.

### DISCUSSION

Bacteriocins have long been recognized as highly specific antibiotics that bacteria develop to outcompete closely related strains. It has also been long thought that these small proteins act by porating bacterial membranes like other pore-forming toxins, some antibiotics and host-defense peptides.8 However, direct evidence for bacteriocin-promoted poration has been lacking, despite the fact that bacteriocins belong to a distinctive family of host defence molecules with a common protein fold.^{3,7,8} Although several bacteriocin structures have been solved,^{12,21,34} the way their structural features specify antimicrobial mechanisms remains obscure. This study partially filled this gap by solving the fold of an archetypal bacteriocin, epidermicin NI01, and correlating it with a unique mechanism comprising several distinctive modes of membrane disruption, in contrast to alternative scenarios that assume one poration mode per membrane-disrupting agent. Furthermore, we experimentally demonstrated that it is the cooperativity of structural constituents, helical hairpins, which orchestrates multiple modes into one synergistic process. For example, the central hairpin, α2α3, was found to have a direct and reciprocal impact on the terminal helices translating different disruption modes into one dynamic process. This mechanism is conserved, favors anionic membranes and is not stereoselective. Our results revealed that the four-helix bundle organisation of bacteriocins is necessary to complete such a highly regulated and sophisticated mechanism. The fold itself encodes this decisively physical means of selective membrane attack that is likely to hold true for other single-chain bacteriocins. The analogous behaviour of another four-helix bacteriocin, A53, supports this conclusion.

Four-helix folds may better adapt to overcome a wide range of resistant membranes. The subtlety with which constituent helices cooperate is what makes bacteriocins less susceptible to acquired antibacterial resistance. This contrasts with host-defense peptides and membrane-active antibiotics that rely on a single disruption mode and are less fit against emerging strategies of membrane resistance.³⁵

### Data and Code Availability

The data supporting the findings of this study is available within Supplemental Information. Coordinates and structure factors were deposited in PDB with the accession codes 6SIF (P21212) and 6SIG (C222).

### Supplemental Information

### Flowering Poration—a Synergistic Multi-Mode Antibacterial Mechanism by a Bacteriocin Fold

### Methods

Polypeptide Synthesis, Identification and Purification. NI01 and all its derivatives were assembled in a Liberty microwave peptide synthesizer (CEM Corp.) using Fmoc/tBu synthesis protocols with DIC/Oxyma as coupling reagents. NI01 and all-D NI01 were assembled on Fmoc-Ala-Wang resin and Fmoc-D-Ala-Wang resins, respectively. Both proteins were capped at their Ntermini using p-nitrophenylformate. All the hairpins were synthesised as C-terminal amides on a Tentagel S RAM resin, leaving the N-termini uncapped. NI01, D-NI01, R-NI01, A53 and hl were cleaved and deprotected using cleavage mixture A (94% TFA, 2% TIS, 2% DODT, 2% H2O). For all the others a mixture B (95% TFA, 2.5% TIS, 2.5% H2O) was used. NI01, D-NI01 and A53 were formylated at their N-termini. All peptides were then purified by semi-preparative RP-HPLC. The purity and identities of NI01 and derivatives were confirmed by analytical RP-HPLC (≥95%) and MALDI-ToF mass-spectrometry: MS [M+H]+: NI01—m/z 6072.3 (calc.), 6072.8 (found); D-NI01—m/z 6072.3 (calc.), 6073.5 (found); R-NI01—m/z 6314.4 (calc.), 6316.3 (found); A53—m/z 6012.5 (calc.), 6013.6 (found); α1α2—m/z 2773.4 (calc.), 2772.8 (found); α2α3—m/z 2383.8 (calc.), 2384.0 (found); α3α4—m/z 3149.7 (calc.), 3150.8 (found); α1α2α3—m/z 3964.8 (calc.), 3964.8 (found); α2α3α4—m/z 4263.0 (calc.), 4263.8 (found); R-NI01—m/z 4263.0 (calc.), 4263.8 (found).

Analytical and semi-preparative RP-HPLC was performed on a Thermo Scientific Dionex HPLC System (Ultimate 3000) using a Vydac C18 analytical and semi-preparative (both 5 μm) columns. Analytical runs used a 10-70% B gradient over 30 min at 1 mL/min, semi-preparative runs were optimised for each peptide, at 4.5 mL/min. Detection was at 280 and 214 nm. Buffer A and buffer B were 5% and 95% (v/v) aqueous CH3CN containing 0.1% TFA.

Crystal structure determination. Crystals of NI01 were obtained in two different forms, P21212 and C222, and diffraction data were collected to resolutions of 1.69 and 1.58 Å, respectively (Table S1). NI01 was obtained in two different crystal forms. The structure of the P21212 crystal form was solved by SIR using phasing from iodide ions. The asymmetric unit (AU) contains 8 NI01 molecules, arranged in 222 symmetry (FIG. 1B); an individual NI01 structure was used to solve the C222 crystal form, which has 4 molecules in the AU. Some of the intramolecular contacts between monomers are preserved between the two crystal forms.

Crystals were grown by sitting drop vapor diffusion at 20° C.: equal volumes (200 nL) were mixed of protein and a reservoir solution of either 0.2 M aq. (NH₄)₂SO₄, 0.1 M aq. CH₃COO-Na+ (pH 4.5), 28% PEG, 2000 MME (P21212 crystal form) or 0.2 M aq. Li₂SO₄, 0.1 M aq. CH₃COO-Na+ (pH 4.5), 24% PEG 8000 (C222 crystal form). Native crystals were cryoprotected by addition of glycerol to 20% (v/v) to liquor from a sitting drop well (all components therefore are at 80% of initial concentrations). Phasing was obtained from soaking of a single P21212 crystal in 0.4 M KI/20% glycerol. The crystal started to dissolve at this KI concentration, but exposure was sufficient to allow recovery with I-ions incorporated. Data were collected at the Diamond Light Source (National Synchrotron Facility, Oxford, UK), using the following beamlines and wavelengths: native P21212 DLS IO4 (1.0725 Å); KI derivative P21212 DLS I04 (1.5000 Å); native C222 DLS I04-1 (0.9200 Å). Data were processed using XDS,³⁶ from within the xia2 system for automated data reduction.37 Space-group assignment was assisted using POINTLESS.³⁸ The KI dataset gave an anomalous slope of 1.13; 28 iodine sites were located using SHELX39 and subsequently phased using BP340 from within CCP4 suite41 to give an FOM of 36% to 2.10 Å.

Electron density maps were improved using SOLOMON42 and a near-complete model for eight separate chains built using BUCCANEER.⁴³ The model was completed by minor manual rebuilding using COOT44 and refinement using REFMAC.⁴⁵ The C222 crystal form was solved with a monomer from chain A of the P21212 crystal form, using PHASER,⁴⁶ as implemented within PHENIX,47 followed by automated model building and refinement in PHENIX. The final structures contained no Ramachandran outliers. Stereochemical parameters for both structures were examined using PROCHECK,⁴⁸ and were within or better than the tolerance limits expected for each structure at the resolution limits given in Table S1.

Lipid Vesicle Preparation. 1-palmitoyl-2-oleoyl-glycero-3-phosphocholine (POPC) with 1-palmitoyl-2-oleoyl-sn-glycero-3-phospho-(1′-rac-glycerol) (POPG) lipids used for vesicle construction were from Avanti Polar Lipids (Alabaster, USA). POPC was used as mammalian model membranes, and POPC/POPG (3:1, molar ratios) was used as bacterial model membranes. The lipids were weighted up, dissolved in chloroform-methanol (2:1, vol/vol), and dried under a nitrogen stream to form a thin film. The film was hydrated in 10 mM phosphate buffer (pH 7.4), vortexed for 2 min and bath sonicated for 30 min. The obtained suspension was extruded using a hand-held extruder (Avanti Polar lipids) (29 times, polycarbonate filter, 0.05 μm) to give a clear solution of small unilamellar vesicles, which were analysed (50 nm) by photon correlation spectroscopy (ZEN3600; Malvern Instruments, UK) following the re-suspension of vesicles to a final concentration of 1 mg/mL. Dynamic light scattering batch measurements were carried out in a low volume disposable cuvette at 25° C. Hydrodynamic radii were obtained through the fitting of autocorrelation data using the manufacturer's Dispersion Technology Software (version 5.10).

Dynamic Light Scattering. Zetasizer Nano (ZEN3600, Malvern Instruments, UK) was used to measure size distributions and ζ-potential in low volume disposable cuvettes and folded capillary cells, respectively. The measurements were performed at 25° C. for NI01 (900 μM) in 10 mM phosphate buffer (pH 7.4). Hydrodynamic radii and ζ-potential values were obtained through the fitting of autocorrelation data using the manufacture's software, Zetasizer Software (version 7.03).

The ζ-potential value reported is a mean of three independent measurements, with each measurement consisting of 10 recordings. Size distributions represent a mean of three independent measurements, with each measurement consisting of 20 recordings.

Circular Dichroism Spectroscopy. Aqueous peptide solutions (300 μL, at a given concentration) were prepared in filtered (0.22 μm), 10 mM phosphate buffer, pH 7.4. CD spectra recorded in the presence of synthetic membranes are for L/P molar ratio of 100. All CD spectra were recorded on a JASCO J-810 spectropolarimeter fitted with a Peltier temperature controller. All measurements were taken in ellipticities in mdeg and converted to molar ellipticities by normalizing for the concentration of peptide bonds and cuvette path length ([θ], deg cm2 dmol−1 res−1). The data collected with a 1 nm step and 1 s collection time per step are presented as the average of 4 scans. Thermal denaturation curves were recorded with 2° C. intervals using 1 nm bandwidth, 180 s equilibration time for each spectrum and with 2° C./min ramp rate.

Isothermal Titration calorimetry. Measurements were obtained using a Microcal isothermal titration calorimeter-200 (ITC-200) which has a cell volume of ˜0.2026 mL and a syringe volume of ˜0.04 mL. The titrations were performed with a 60-s initial delay and a 120-s equilibration time between the start and end of each titration. Experiments were performed at 30° C. with a stirring speed at 750 rpm until no further enthalpy changes were observed. Binding isotherms were recorded for NI01 (500 μM, 38 injections of 1 μL each) titrated into lipid vesicles (380 μM, total lipid) in the cell. The observed heats were corrected for dilution effects by titrating the protein into the buffer. All data were corrected for the volume of the added titrant and analysed by proprietary software (Microcal Origin 7.0) using one-set binding model to allow for the determination of association constants (Ka), changes in enthalpy (ΔH) and entropy (ΔS). Each experiment was performed in duplicate

Preparation of SLBs for in-liquid AFM imaging. SLBs were formed using a vesicle fusion method as described elsewhere.¹⁹ Freshly prepared vesicles (1.5 μL, 3 mg/mL) were added to cleaved mica that was pre-hydrated in 20 mM MOPS, 120 mM NaCl, 20 mM MgCl2 (pH 7.4).

After incubation over 45 min, the samples were washed 10 times with imaging buffer (20 mM MOPS, with 120 mM NaCl, pH 7.4) to remove unfused vesicles. The resulting SLBs were checked to confirm they were defect free. Mica discs (Agar Scientific, Stansted, UK) were glued to a metal puck, and freshly cleaved prior to lipid deposition.

In-liquid AFM imaging of SLBs. The topographic imaging of SLBs in aqueous buffers was performed on a Multimode 8 AFM system (Bruker AXS, USA) using Peak Force Tapping™ mode and MSNL-E cantilevers (Bruker AFM probes, USA). Images were taken at the PeakForce frequency of 2 kHz, PeakForce amplitude of 10-20 nm and PeakForce set-point of 10-30 mV (<100 pN). The images were then processed using Gwyddion (http://gwyddion.net) for line-by-line background subtraction (flattening) and plane fitting. NI01 or its derivatives were introduced into a 100-μL fluid cell (Bruker AXS, USA) to the final concentrations stated.

Minimum Inhibitory Concentrations assay. Minimum inhibitory concentrations (MICs) were determined by broth microdilution on P. aeruginosa, E. coli, S. aureus, M. luteus, B. subtilis, S. typhimurium and K. pneumoniae according to the Clinical and Laboratory Standards Institute. Typically, 100 μL of 0.5-1×106 CFU per ml of each bacterium in Mueller Hinton media broth (Oxoid) were incubated in 96-well microtiter plates with 100 μL of serial two-fold dilutions of the corresponding antimicrobial agent (from 100 to 0 μM) at 37° C. on a 3D orbital shaker. The absorbance was measured after the addition of NI01, its derivatives or an antibiotic at 600 nm using a SpectraMax i3× Multi-Mode Microplate Reader (Molecular Devices). MICs were defined as the lowest protein concentration that inhibited visible bacterial growth after 24 h at 37° C. All tests were done in triplicate and results are summarized in Tables S2 and S3.

Hemolysis assay. Hemolysis was determined using human erythrocytes sourced commercially from Cambridge Bioscience Ltd. and used within two days. 10% (vol/vol) suspensions of human erythrocytes were incubated with NI01, its derivatives or antibiotics. The cells were rinsed four times in 10 mM phosphate buffer saline (PBS, GibcoTM), pH 7.2, by repeated centrifugation and re-suspension (3 min at 3000×g). The cells were then incubated at room temperature for 1 h in either deionized water (fully hemolysed control), PBS, or with a corresponding antimicrobial agent in PBS. After centrifugation at 10,000×g for 5 min, the supernatant was separated from the pellet, and the absorbance was measured at 550 nm using a SpectraMax i3× Multi-Mode Microplate Reader (Molecular Devices). Absorbance of the suspension treated with deionized water defined complete hemolysis. All tests were done in triplicate and results are shown in Table S3. The values given in Table S2 correspond to concentrations needed to lyse half of the sample population (50% lysis of erythrocytes) and are expressed as median hemolytic doses—HD50.

Table S1. X-ray data collection and refinement statistics.

Table S2. Biological activities of NI01, its derivatives and other antimicrobial agents for comparison.

Table S3. Antibacterial activities of NI01 and its derivatives.

FIG. 6. Post-synthetic characterisation. MALDI-ToF mass spectrometry spectra for purified NI01 and NI01 derivatives used in the study.

FIG. 7. NI01 folding monitored by CD spectroscopy. CD spectra for (A) NI01 recorded before (black line) and after (red line) thermal denaturation; (B) NI01 recorded at 2° C. intervals during the thermal unfolding from 20° C. to 90° C.; (C) NI01 at varied TFE concentrations; (D) NI01 at different protein concentrations. Folding conditions: 20 μM protein, pH 7.4, 10 mM phosphate buffer, 20° C.

FIG. 8. NI01 monodispersity in solution. (A) Size distributions by dynamic light scattering by number and volume for NI01 (0.9 mM) in 10 mM phosphate buffer, pH 7.4. (B) Correlograms showing rapid correlation decreases from high intercepts, which is characteristic of monodisperse, small particles.

FIG. 9. NI01 interactions with reconstituted phospholipid membranes. (A) CD spectra for NI01 (upper) and its all-D form (lower) (20 μM) in phosphate buffer (black line) and in anionic (blue line) and zwitterionic (red line) membranes at 100 lipid/protein (L/P) ratios. (B) Isothermal titration calorimetry of NI01 (0.5 mM) binding to mammalian mimetic membranes. Heat absorbed (μcal/s) for each isotherm is plotted versus titration time (upper panel). Integrated heats (kcal/mol) are plotted versus protein-lipid molar ratios (lower panel).

FIG. 10. In-liquid AFM imaging of reconstituted phospholipid membranes incubated with NI01. (A) Topography of SLBs mimicking bacterial membranes treated with NI01 (0.25 μM). (B) Topography micrographs of SLBs mimicking mammalian membranes treated with NI01 at higher concentrations (0.6 μM), with height profile as measured along the white, dashed line. Colour and length scale bars are 15 nm and 500 nm, respectively.

FIG. 11. A boomerang-like shape of the central hairpin, α2α3. Two different points of view are given to show that α2 and α3 are linked at an obtuse angle (left) forming a flat conformation (right).

FIG. 12. Interactions of two-helix hairpins with reconstituted phospholipid membranes. CD spectra for the hairpins (20 μM) in phosphate buffer (black line) and in anionic (blue line) and zwitterionic (red line) membranes at 100 lipid/protein (L/P) ratios.

FIG. 13. Three-helix hairpins. (A) In-liquid AFM imaging of SLBs mimicking bacterial membranes incubated with the hairpins (0.25 μM). The images were taken within the first 5 min of incubation. Height profiles measured along the highlighted lines. Colour scale bar is 15 nm. Length scale bars are 500 nm (left) and 200 nm (right). (B) CD spectra for the hairpins (20 μM) in phosphate buffer (black line) and in anionic (blue line) and zwitterionic (red line) membranes at 100 lipid/protein (L/P) ratios.

FIG. 14. Cooperative structural arrangements of H25. (A) A polar cluster at H25 in the central kink hosting a binding site for SO2-4. (B) Cooperative positioning of the residue forming an aromatic π-π pair with W50.

FIG. 15. Arginine NI01 mutant, R-NI01. (A) In-liquid AFM imaging of SLBs mimicking bacterial membranes incubated with the mutant (0.25 μM). The images were taken within the first 5 min of incubation. Height profiles measured along the highlighted lines. Colour scale bar is 15 nm. Length scale bars are 500 nm (left) and 200 nm (right). (B) CD spectra for the mutant (20 μM) in phosphate buffer (black line) and in anionic (blue line) and zwitterionic (red line) membranes at 100 lipid/protein (L/P) ratios.

FIG. 16. In-liquid AFM imaging of reconstituted bacterial membranes incubated with all-D NI01. (A) Topography of SLBs treated with D-NI01 (0.25 μM), with low-magnification (left) and high magnification (right) images taken within the first 5 min of incubation. (B) A high-magnification image with height profiles as measured along the blue and white dashed lines. Colour bar is 15 nm, length scale bars are 500 nm for (A, left) and 100 nm for (A, right) and B.

FIG. 17. A53 folding. (A) thermal unfolding curve and its first derivative highlighting a single transition point (TM). CD spectra (B) recorded before (black line) and after (red line) thermal denaturation; (C) at different protein concentrations and (D) at varied TFE concentrations. Folding conditions: 20 μM protein, pH 7.4, 10 mM phosphate buffer, 20° C.

The present inventions can be embodied in other specific apparatus and/or methods. The described embodiments are to be considered in all respects as illustrative and not restrictive. In particular, the scope of the invention is indicated by the appended claims rather than by the description and figures herein. All changes that come within the meaning and range of equivalency of the claims are to be embraced within their scope.

### REFERENCES

1. Parker, M. W., and Feil, S. C. (2005). Pore-forming protein toxins: from structure to function. Prog. Biophys. Mol. Biol. 88, 91-142.
2. Koehbach, J., and Craik, D. J. (2019). The vast structural diversity of antimicrobial peptides. Trends

Pharmacol. Sci. 40, 517-528.
3. Cotter, P. D., Ross, R. P., and Hill, C. (2013). Bacteriocins—a viable alternative to antibiotics? Nat. Rev. Microbiol. 11, 95-105.
4. Iacovach, I., Bischofberger, M., and van der Goot, F. G. (2010). Structure and assembly of pore-forming proteins. Curr. Opin. Struct. Biol. 20, 241-216.
5. Lazar, V., Martins, A., Spohn, R., Daruka, L., Grézal, G., Fekete, G., Számel, M., Jangir, P. K., Kintses, B., Csörgő, B., et al. (2018). Antibiotic-resistant bacteria show widespread collateral sensitivity to antimicrobial peptides. Nat. Microbiol. 3, 718-731.
6. Pfeil, M. P., Pyne, A. L. B., Losasso, V., Ravi, J., Lamarre, B., Faruqui, N., Alkassem, H., Hammond, K., Judge, P. J., Winn, M., et al. (2018). Tuneable poration: host defense peptides as sequence probes for antimicrobial mechanisms. Sci. Rep. 8, 14926.
7. Acedo, J. Z., Chiorean, S., Vederas, J. C., and van Belkum, M. J. (2018). The expanding structural variety among bacteriocins from Gram-positive bacteria. FEMS Microbiol. Rev. 42, 805-828.
8. Hechard, Y., and Sahl, H. G. (2002). Mode of action of modified and unmodified bacteriocins from Gram positive bacteria. Biochimie 84, 545-557.
9. Arnison, P. G., Bibb, M. J., Bierbaum, G., Bowers, A. A., Bugni, T. S., Bulaj, G., Camarero, J. A., Campopiano, D. J., Challis, G. L., Clardy, J., et al. (2013). Ribosomally synthesized and post-translationally modified peptide natural products: overview and recommendations for a universal nomenclature. Nat. Prod. Rep. 30, 108-160.
10. Cotter, P. D., Hill, C., and Ross, R. P. (2005). Bacteriocins: developing innate immunity for food. Nat. Rev. Microbiol. 3, 777-788.
11. Sandiford, S., and Upton, M. (2012). Identification, characterization, and recombinant expression of epidermicin NI01, a novel unmodified bacteriocin produced by Staphylococcus epidermidis that displays potent activity against Staphylococci. Antimicrob. Agents Chemother. 56, 1539-1547.
12. Lohans, C. T., Towle, K. M., Miskolzie, M., McKay, R. T., van Belkum, M. J., McMullen, L. M., and Vederas, J. C. (2013). Solution structures of the linear leaderless bacteriocins enterocin 7A and 7B resemble carnocyclin A, a circular antimicrobial peptid e. Biochemistry 52, 3987-3994.
13. Krissinel, E., and Henrick, K. (2007). Inference of macromolecular assemblies from crystalline state. J. Mol. Biol. 372, 774-797.
14. Kelly, S. M., Jess, T. J., and Price, N. C. (2005). How to study proteins by circular dichroism. Biochim. Biophys. Acta, Proteins Proteomics 1751, 119-139.
15. Roccatano, D., Colombo, G., Fioroni, M., and Mark, A. E. (2002). Mechanism by which 2,2,2-trifluoroethanol/water mixtures stabilize secondary-structure formation in peptides: A molecular dynamics study. Proc. Natl. Acad. Sci. USA 99, 12179-12184.
16. Seelig, J. (2004). Thermodynamics of lipid-peptide interactions. Biochim. Biophys. Acta, Biomembranes 1666, 40-50.
17. Khatib, T. O., Stevenson, H., Yeaman, M. R., Bayer, A. S., and Pokorny, A. (2016). Binding of daptomycin to anionic lipid vesicles is reduced in the presence of lysylphosphatidylglycerol.
Antimicrob. Agents Chemother. 60, 5051-5053.
18. Rakowska, P. D., Jiang, H., Ray, S., Pyne, A.; Lamarre, B., Carr, M., Judge, P. J., Ravi, J., Gerling, U. I., Koksch, B., et al. (2013). Nanoscale imaging reveals laterally expanding antimicrobial pores in lipid bilayers. Proc. Natl. Acad. Sci. USA, 110, 8918-8923.
19. Lin, W.-C., Blanchette, C. D., Ratto, T. V., and Longo, M. L. (2006). Lipid asymmetry in DLPC/DSPC-supported lipid bilayers: a combined AFM and fluorescence microscopy study. Biophys. J. 90, 228-237.
20. Mingeot-Leclercq, M.-P., Deleu, M., Brasseur, R., and Dufrene, Y. F. (2008). Atomic force microscopy of supported lipid bilayers. Nat. Protoc. 3, 1654-1659.
21. González, C., Langdon, G. M., Bruix, M., Gálvez, A., Valdivia, E., Maqueda, M., and Rico, M. (2000). Bacteriocin AS-48, a microbial cyclic polypeptide structurally and functionally related to mammalian NK-lysin. Proc. Natl. Acad. Sci. USA 97, 11221-11226.
22. Michalek, M., Sonnichsen, F. D., Wechselberger, R., Dingley, A. J., Hung, C. W., Kopp, A., Wienk, H., Simanski, M., Herbst, R., Lorenzen, I. et al. (2013). Structure and function of a unique pore-forming protein from a pathogenic acanthamoeba. Nat. Chem. Biol., 9, 37-42.
23. Heath, G. R., Harrison, P. L., Strong, P. N., Evans, S. D., and Miller, K. (2018). Visualization of diffusion limited antimicrobial peptide attack on supported lipid membranes. Soft Matter, 14, 6146-6154.
24. Mecke, A., Lee, D. K., Ramamoorthy, A., Orr, B. G., and Banaszak Holl, M. M. (2005). Membrane thinning due to antimicrobial peptide binding: an atomic force microscopy study of MSI-78 in lipid bilayers. Biophys. J, 89, 4043-4050.
25. Jang, H., Ma, B., Woolf, T. B., and Nussinov, R. (2006). Interaction of protegrin-1 with lipid bilayers: membrane thinning effect. Biophys. J, 91, 2848-2859.
26. Pyne, A., Pfeil, M. P., Bennett, I., Ravi, J., Iavicoli, P., Lamarre, B., Roethke, A., Ray, S., Jiang, H., Bella, A. et al. (2017). Engineering monolayer poration for rapid exfoliation of microbial membranes. Chem. Sci. 8, 1105-1115.
27. Li, L., Vorobyov, I. and Allen, T. W. (2013). The different interactions of lysine and arginine side chains with lipid membranes. J Phys Chem B 117, 11906-11920.
28. Yeaman, M. R., and Yount, N. Y. (2003). Mechanisms of antimicrobial peptide action and resistance. Pharmacol. Rev. 55, 27-55.
29. Tocheva, E. I., López-Garrido, J., Hughes, H. V., Fredlund, J., Kuru, E., Vannieuwenhze, M. S., Brun, Y. V., Pogliano, K., and Jensen, G. J. (2013). Peptidoglycan transformations during Bacillus subtilis sporulation. Mol. Microbiol. 88, 673-686.
30. Vollmer, W. Structural variation in the glycan strands of bacterial peptidoglycan. (2008). FEMS Microbiol. Rev. 32, 287-306.
31. Garcia, A. B., Viñuela-Prieto, J. M., López-Gonzalez L., and Candel, F. J. (2017). Correlation between resistance mechanisms in Staphylococcus aureus and cell wall and septum thickening. Infect Drug Resist. 10, 353-356.
32. Ebbensgaard, A., Mordhorst, H., Aarestrup, F. M., and Hansen, E. B. (2018). The role of outer membrane proteins and lipopolysaccharides for the sensitivity of Escherichia coli to antimicrobial peptides. Front Microbiol. 9, 2153.
33. Hammond, K., Lewis, H., Faruqui, N., Russell, C., Hoogenboom, B. W., and Ryadnov, M. G. (2019). Helminth defense molecules as design templates for membrane active antibiotics. ACS Infect Dis. 5, 1471-1479.
34. Acedo, J. Z., van Belkum, M. J., Lohans, C. T., Towle, K. M., Miskolzie, M., and Vederas, J. C. (2016). Nuclear magnetic resonance solution structures of lacticin Q and aureocin A53 reveal a structural motif conserved among leaderless bacteriocins with broad spectrum activity. Biochemistry 55, 733-742.
35. Needham, B. D., and Trent, M. S. (2013). Fortifying the barrier: the impact of lipid A remodeling on bacterial pathogenesis. Nat. Rev. Microbiol. 11, 467-481.
36. Kabsch, W. (2010). XDS. Acta Crystallogr. D Biol. Crystallogr. 66, 125-132.
37. Winter, G. xia2: an expert system for macromolecular crystallography data reduction. (2010). J Appl. Crystallogr. 43, 186-190.
38. Evans, P. (2006). Scaling and assessment of data quality. Acta Crystallogr. D Biol. Crystallogr. 62, 72-82.
39. Sheldrick, G. (2008). A short history of SHELX. Acta Crystallogr. A 64, 112-122.
40. Pannu, N. S., Waterreus, W.-J., Skubak, P., Sikharulidze, I., Abrahams, J. P., and de Graaff, R. A. G. (2011). Recent advances in the CRANK software suite for experimental phasing. Acta Crystallogr. D Biol. Crystallogr. 67, 331-337.
41. Collaborative Computational Project, Number 4. (1994). The CCP4 Suite: Programs for protein crystallography. Acta Crystallogr. D Biol. Crystallogr. 50, 760-763.
42. Abrahams, J. P., and Leslie, A. G. W. (1996). Methods used in the structure determination of bovine mitochondrial F1 ATPase. Acta Crystallogr. D Biol. Crystallogr. 52, 30-42.
43. Cowtan, K. (2006). The Buccaneer software for automated model building. 1. Tracing protein chains. Acta Crystallogr. D Biol. Crystallogr. 62, 1002-1011.
44. Emsley, P., Lohkamp, B., Scott, W. G., and Cowtan, K. (2010). Features and development of Coot. Acta Crystallogr. D Biol. Crystallogr. 66, 486-501.
45. Murshudov, G. N., Vagin, A. A., and Dodson, E. J. (1997). Refinement of Macromolecular Structures by the Maximum Likelihood Method. Acta Crystallogr. D Biol. Crystallogr. 53, 240-255.
46. McCoy, A. J., Grosse-Kunstleve, R. W., Adams, P. D., Winn, M. D., Storoni, L. C., and Read, R. J. (2007). Phaser crystallographic software. J Appl. Crystallogr. 40, 658-674.
47. Adams, P. D., Afonine, P. V., Bunkoczi, G., Chen, V. B., Davis, I. W., Echols, N., Headd, J. J., Hung, L.-W., Kapral, G. J., Grosse-Kunstleve, R. W. et al. (2010). PHENIX: a comprehensive Python-based system for macromolecular structure solution. Acta Crystallogr. D Biol. Crystallogr. 66, 213-221.
48. Laskowski, R. A., McArthur, M. W., Moss, D. S., and Thornton, J. M. (1993) PROCHECK: a program to check the stereochemical quality of protein structures. J Appl. Crystallogr. 26, 283-291.

TABLE S1
X-ray data collection asnd refinement statistics

| | Native | Iodine soak | Native |
|---|---|---|---|
| Data collection | | | |
| Space group | P 2, 2, 2 | P 2, 2, 2 | C222 |
| Cell dimensions (Å) | 92.2, 1179, 52.9 | 92.7, 117.6, 52.4 | 90.7, 99.4, 69.4 |
| Resolution (Å) | 46-1.69 (1.73- | 93-2.10 (2.15- | 49-1.58(1.62- |
| | 1.69)¹* | 2.10) [symbol] | 1.58) [symbol] |
| R_{merge} (%) | 5.8 (57.6) | 11.3 (66.7) | 5.4 (64.9) |
| I/σI | 14.1 (2.1) | 15.4 (2.3) | 18.3 (2.6) |
| Completeness (%) | 99.0 (97.6) | 96.8 (76.5) | 99.9 (100) |
| Redundancy | 4.4 (4.2) | 10.6 (4.6) | 6.4 (6.3) |
| Refinement | | | |
| Resolution (Å) | 46-1.69 | | 49-1.58 |
| No. reflections | 61,278 | | 40.821 |
| R_{work}/R_{free} | 0.247/0.262 | | 0.157/0.174 |
| No. atoms | | | |
| Protein | 3,374 | | 1.706 |
| Ligand/ion | 40 | | 10 |
| Water | 103 | | 231 |
| B-factors (Å²) | | | |
| Protein | 24.6 | | 21.6 |
| Ligand/ion | 39.0 | | 27.8 |
| Water | 26.4 | | 36.5 |
| R.m.s deviations | | | |
| Bond lengts (Å) | 0.010 | | 0.012 |
| Bond angles (°) | 1.47 | | 1.70 |

*Values in parentheses are for highest-resolution shell
¹Data were collected from a single crystal [symbol]
indicates data missing or illegible when filed

TABLE S2
Biological activities of NI01, its derivatives and other antimicrobial agents for comparison.

| | Antimicrobial agent | | | | | | | | | | |
|---|---|---|---|---|---|---|---|---|---|---|---|
| | NI01 | | | | | | | | | | |
| Cell | L-form | D-form | R-mutant | α1α2 | α2α3 | α3α4 | α1α2α3 | α2α3α4 | ampicillin | melittin | polymyxin B |
| | Minimum Inhibitory Concentration, μg/mL | | | | | | | | | | |
| E. coli (ATCC 15597) | 18 | 18 | 9 | 8 | 14 | 19 | 6 | 6 | 24 | 7 | 2 |
| S. aureus (ATCC 6538) | 5 | 5 | 5 | 8 | >120 | 5 | 6 | 6 | 1 | 3 | 32 |
| S. typhimurium (DA6192) | >300 | >300 | >300 | 32 | >50 | 40 | 20 | 12 | 8 | 9 | 2 |
| B. subtilis (ATCC 6633) | 3 | 3 | 18 | 16 | 14 | 3 | 6 | 6 | 9 | 9 | 4 |
| K. pneumoniae (NCTC 5055) | >300 | >75 | 18 | 16 | 28 | 40 | 20 | 12 | 5 | 9 | 4 |
| M. luteus (ATCC 49732) | 2 | 2 | 2 | 3 | 2 | 9 | 6 | 3 | 1 | 2 | 2 |
| P. aeruginosa (ATCC 27853) | >300 | >300 | >300 | >140 | 14 | >80 | 12 | 6 | 8 | 25 | 2 |
| | HD₃₀,^{a} μg/mL | | | | | | | | | | |
| Human erythrocytes | 500 | 600 | 250 | 150 | UD^{b} | 150 | 200 | 200 | UD^{b} | 5 | 350 |

^{a}median hemolytic doses to achieve 50% lysis;
^{b}undetectable

TABLE S3
Antibacterial activities of NI01 and its derivatives.

| | Antimicrobial agent | | | | | | |
|---|---|---|---|---|---|---|---|
| | NI01 | | | | | | |
| | L-form | D-form | α1α2 | α2α3 | α3α4 | α1α2α3 | α2α3α4 |
| Cell | Minimum Inhibitory Concentration, μg/mL | | | | | | |
| EMRSA (12817) | 4 | 4 | 4 | >128 | 4 | 32 | 16 |
| EMRSA (12845) | 4 | 4 | 2 | >128 | 4 | 32 | 16 |
| EMRSA (12873) | 4 | 8 | 4 | >128 | 4 | 32 | 16 |
| E. coli (SBS363) | 8 | 8 | 1 | 8 | 4 | 4 | 4 |
| E. coli (ML35) | 64 | 64 | 8 | 32 | 32 | 64 | 32 |

1. An isolated or recombinant polypeptide comprising SEQ ID NO: 2 or having at least 75% identity thereto, wherein the isolated or recombinant polypeptide is bactericidal and/or bacteriostatic.

2. A polypeptide according to claim 1, having at least 90% identity to SEQ ID NO: 2.

3-11. (canceled)

12. An isolated or recombinant nucleic acid sequence comprising a sequence encoding the polypeptide according to claim 1.

13. A pharmaceutical composition comprising the polypeptide of claim 1.

14. A pharmaceutical composition according to claim 13 for the treatment of a bacterial infection.

15. An anti-microbial formulation comprising the polypeptide according to claim 1.

16. The isolated or recombinant polypeptide of claim 1, consisting of SEQ ID NO: 2.
